SEQUENCE LISTING (1 sequence)

SEQ ID NO: 1 - DNA, 2540 bp, synthetic construct
  source 1..2540: mol_type = other DNA
ggtacccgagggcagaggaagtcttctaacatgcggtgacgtggaggagaatcccggccctaggcttgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgagggatcccatgggccgcgccggctctagatcgcgaacgcgtgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagtcgaaattctctagagctcgctgatcagcctcgactgtgccttctagttgccagccatctgttgtttgcccctccccccgtgccttccttgaccctggaaggtgccactcccactgtcctttcctaataaaatgaggaaattgcatcgcattgtctgagtaggtgtcatcgacgtcttcccctagagttctgtgactaggctcccatatggaacaaccatattctttgaggggtcactggctttaggacagggaggccagggaagaggtgg

(19) United States
(12) Patent Application Publication
(10) Pub. No.: US 2026/0265686 A1
(43) Pub. Date: Sep. 10, 2026

(54) HUMAN PLURIPOTENT CELL LINE GENETICALLY ENGINEERED TO CO-EXPRESS alpha-MHC GENE AND FLUORESCENT REPORTER GENE

(71) Applicants: CATHOLIC KWANDONG UNIVERSITY INDUSTRY FOUNDATION, Gangneung-si, Gangwon-do (KR); SEOUL NATIONAL UNIVERSITY R&DB FOUNDATION, Seoul (KR)

(72) Inventors: Ji Ho JANG, Seoul (KR); Young Min CHOI, Seoul (KR); Jong Kwan JUN, Seoul (KR); Hye Won SEOL, Seoul (KR); Ju Won JUNG, Seoul (KR)

(21) Appl. No.: 18/847,280

(22) PCT Filed: Mar. 14, 2023
(86) PCT No.: PCT/KR2023/003397

§ 371 Date: Sep. 16, 2024

(30) Foreign Application Priority Data
Mar. 16, 2022 (KR) 10-2022-0032828

(51) Int. Cl. C12N 5/0735; C07K 14/435; C07K 14/47; C12N 5/077; G01N 15/01; G01N 15/10; G01N 15/1434

(52) U.S. Cl. CPC C12N 5/0606; C07K 14/43595; C07K 14/47; C12N 5/0657; G01N 15/01; G01N 15/1434; C12N 2506/02; C12N 2510/00; G01N 2015/1006

(57) ABSTRACT

Provided is a genetically engineered human pluripotent cell line wherein an insert sequence including a nucleotide sequence of a fluorescent reporter gene is inserted following a stop codon for the α-MHC gene, whereby the α-MHC gene and the fluorescent reporter gene are co-expressed. By genetically manipulating the cell line to express the fluorescent reporter gene upon expression of the α-MHC gene, which is a myocardial cell marker, it is possible to monitor the differentiation process by observing the fluorescent signal when the cell line is differentiated into myocardial cells.

### TECHNICAL FIELD

The present invention relates to a pluripotent cell line into which a reporter gene has been inserted by genetic engineering to monitor the expression of the α-MHC gene, a cardiomyocyte marker.

### BACKGROUND ART

Stem cells are cells that have a unique ability (multipotentiality) to produce many different types of cells, and can regenerate cells in damaged parts of the body. The term “stem cell” comes from the word “stem”, meaning to originate, and in Japan, it is also called a stem cell, meaning a stem that comes from the progenitor cell or root.

It is the general consensus that the potency of stem cells decreases with their development from the fertilized egg cell to the embryonic stage to the adult organism. According to this property of stem cells, the fertilized egg cell is referred to as totipotent, embryonic stem cells/induced pluripotent stem cells are referred to as pluripotent, and adult stem cells are referred to as multipotent. Here, the totipotent cells are cells from which a complete organism can develop. In addition to the fertilized egg cell, the totipotent cells also include the cells of the early embryonic phase. Stem cells with pluripotency include embryonic stem cells, which are typically obtained from the internal cell mass of disaggregated blastocytes, and induced pluripotent stem cells, which are obtained by reprogramming somatic cells to have pluripotency.

Since pluripotent embryonic stem cells and induced pluripotent stem cells can differentiate into all body cells, many studies have been conducted to differentiate these pluripotent stem cells into desired cell types for use in the treatment of diseases.

Cardiovascular disease is a disease that occurs in the heart and major arteries. Cardiovascular disease is one of the leading causes of death after malignant tumors and its importance is growing, but its incidence rate is continuously increasing. These cardiovascular diseases are caused by loss of myocardium or cause serious damage to the myocardium. However, since myocardium is difficult to self-regenerate, there is no treatment that can fundamentally treat the main disease causes except for heart transplantation. Therefore, to overcome the limitations of conventional treatments, studies have been actively conducted to differentiate stem cells into cardiomyocytes and use them as a cell therapy product. In these differentiation studies, it is very important to monitor whether differentiation into cardiomyocytes is progressing properly.

Accordingly, in order to monitor differentiation into cardiomyocytes, the present inventors have produced a cell line by inserting a fluorescent reporter gene into the α-MHC gene, which is known to be involved in heartbeat generation in cardiomyocytes.

### DISCLOSURE

### Technical Problem

An object of the present invention is to provide a human pluripotent cell line whose differentiation into cardiomyocytes may be monitored.

### Technical Solution

In order to achieve the above object, in one embodiment of the present invention, there is provided a genetically engineered human pluripotent cell line wherein an insertion sequence comprising a fluorescent reporter gene sequence is inserted following a stop codon of an α-MHC gene so that the α-MHC gene and the fluorescent reporter gene are co-expressed.

In one embodiment, the fluorescent reporter may be selected from the group consisting of Sirius, EBFP, ECFP, mTurquoise, TagCFP, AmCyan, mTFP1, MidoriishiCyan, CFP, TurboGFP, AcGFP, TagGFP, Azami-Green, ZsGreen, EmGFP, EGFP, GFP2, HyPer, TagYFP, EYFP, Venus, YFP, PhiYFP, PhiYFP-m, TurboYFP, ZsYellow, mBanana, KusabiraOrange, mOrange, TurboRFP, DsRed-Express, DsRed2, TagRFP, DsRed-Monomer, AsRed2, mStrawberry, TurboFP602, mRFP1, JRed, KillerRed, mCherry, HcRed, KeimaRed, mRasberry, mPlum, PS-CFP, Dendra2, Kaede, EosFP, and KikumeGR.

In one embodiment, the insertion sequence may comprise a T2A cleavage sequence, the fluorescent reporter gene sequence, and a puromycin resistance gene sequence.

In one embodiment, the insertion sequence may comprise SEQ ID NO: 1.

In one embodiment, the α-MHC and fluorescent reporter genes may be translated into their respective proteins by the T2A cleavage sequence.

In one embodiment, the genetic engineering may be achieved by a CRISPR/Cas9 system.

In one embodiment, the human pluripotent cell line may be produced by genetically engineering a human embryonic stem cell line, SNUhES3.

In one embodiment, the genetically engineered human pluripotent cell line may express fluorescence while differentiating into cardiomyocytes.

In another embodiment of the present invention, there is a method of monitoring differentiation into cardiomyocytes using the genetically engineered human pluripotent cell line.

### Advantageous Effects

The cell line produced according to one embodiment of the present invention may provide a method capable of monitoring whether differentiation into cardiomyocytes is progressing properly by checking the fluorescence signal of the fluorescent reporter during differentiation into cardiomyocytes.

Effects of the present invention are not limited to the effects described above, and should be understood to include all effects that may be deduced from the contents described in the description of the invention or the claims.

### BRIEF DESCRIPTION OF DRAWINGS

FIG. 1 shows a gene sequence, which is targeted by the sgRNA of the CRISPR/Cas9 system for genetic engineering of a cell line, and the structure of a donor construct.

FIG. 2 shows the results of PCR analysis of gDNA of a SNUe003-A-3 cell line.

FIG. 3 shows gene sequencing results for the SNUe003-A-3 cell line.

FIG. 4 shows karyotype analysis results for the SNUe003-A-3 cell line.

FIG. 5 shows the results of testing for Mycoplasma infection of the SNUe003-A-3 cell line.

FIG. 6 shows the results of analyzing the morphology and AP activity of the SNUe003-A-3 cell line.

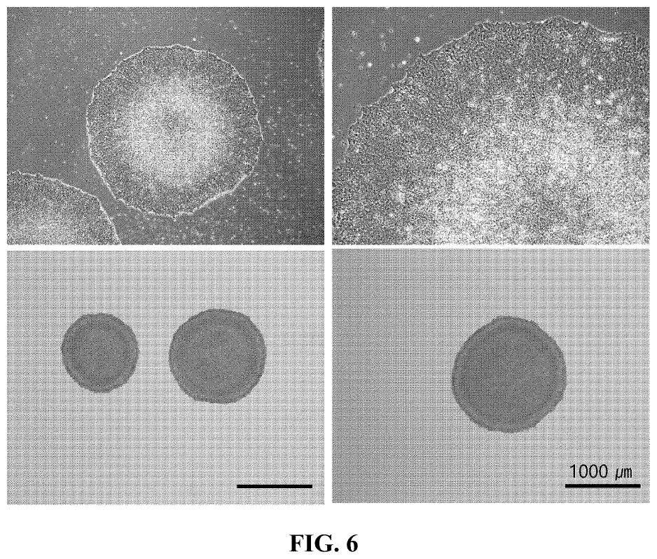

FIG. 7 shows the results of analyzing the expression of pluripotency markers OCT4, SSEA-4, TRA-1-60, and TRA-1-81 in the SNUe003-A-3 cell line through fluorescent staining.

FIG. 8 shows the results of analyzing the expression of pluripotency markers OCT4, SOX2 and NANOG in the SNUe003-A-3 cell line through RT-PCR.

FIG. 9 shows the results of analyzing the expression of markers of three germ layers through fluorescent staining after inducing differentiation of the SNUe003-A-3 cell line into the three germ layers.

FIG. 10 shows the results of examining whether EGFP is expressed after differentiating the SNUe003-A-3 cell line into cardiomyocytes.

FIG. 11 shows the results of analyzing the expression of a cardiomyocyte marker in EGFP-expressing cells through qPCR after differentiating the SNUe003-A-3 cell line into cardiomyocytes.

FIG. 12 shows the results of analyzing whether α-MHC is expressed in EGFP-expressing cells through flow cytometry analysis after differentiating the SNUe003-A-3 cell line into cardiomyocytes.

### BEST MODE

Hereinafter, the present invention will be described in detail.

According to one aspect of the present invention, there is provided a genetically engineered human pluripotent cell line wherein an insertion sequence comprising a fluorescent reporter gene sequence is inserted following a stop codon of an α-MHC gene so that the α-MHC gene and the fluorescent reporter gene are co-expressed.

The α-MHC gene starts to be expressed in the early stage of cardiomyocyte differentiation, plays an important role in differentiation and maturation into cardiomyocytes, continues to be expressed even in adult cardiomyocytes, and is involved in heartbeat generation. Therefore, by checking the expression of the α-MHC gene, it is possible to monitor whether differentiation into cardiomyocytes is proceeding properly.

The fluorescent reporter refers to a fluorescent substance that is used to report whether a specific gene is expressed. Therefore, a general fluorescent substance may be used as the reporter. For example, the fluorescent reporter may be selected from the group consisting of Sirius, EBFP, ECFP, mTurquoise, TagCFP, AmCyan, mTFP1, MidoriishiCyan, CFP, TurboGFP, AcGFP, TagGFP, Azami-Green, ZsGreen, EmGFP, EGFP, GFP2, HyPer, TagYFP, EYFP, Venus, YFP, PhiYFP, PhiYFP-m, TurboYFP, ZsYellow, mBanana, KusabiraOrange, mOrange, TurboRFP, DsRed-Express, DsRed2, TagRFP, DsRed-Monomer, AsRed2, mStrawberry, TurboFP602, mRFP1, JRed, KillerRed, mCherry, HcRed, KeimaRed, mRasberry, mPlum, PS-CFP, Dendra2, Kaede, EosFP, and KikumeGR, without being limited thereto. Preferably, the green fluorescent protein EGFP may be used.

The insertion sequence comprising the fluorescent reporter may be inserted following the stop codon of the α-MHC gene to monitor differentiation into cardiomyocytes. In this case, the fluorescent reporter gene and the α-MHC gene can be expressed as the same mRNA. In order to translate α-MHC and the fluorescent reporter into their respective proteins, an IRES sequence, the 2A system sequence F2A, P2A or T2A cleavage sequence may be located between the stop codon and the fluorescent reporter gene sequence, without being limited thereto. Preferably, the T2A cleavage sequence may be used. Thereby, the α-MHC gene and the fluorescent gene may be co-expressed without affecting the α-MHC function even after genetic engineering.

In addition to the fluorescent reporter gene and the cleavage sequence, the insertion sequence may comprise an antibiotic resistance gene for selecting the genetically engineered cell line. For example, a neomycin, puromycin or hygromycin resistance gene may be used, without being limited thereto. Preferably, the puromycin resistance gene may be used.

For example, since cell line selection using the puromycin resistance gene is performed before differentiation into cardiomyocytes, expression of the puromycin resistance gene may be induced by an independent promoter that can induce expression in pluripotent stem cells rather than being induced by the promoter of the α-MHC gene. For example, the independent promoter may be selected from among CMV, U6, E2F, EF1-alpha and PGK promoters, without being limited thereto. Preferably, the PGK promoter may be used.

The insertion sequence may comprise SEQ ID NO: 1.

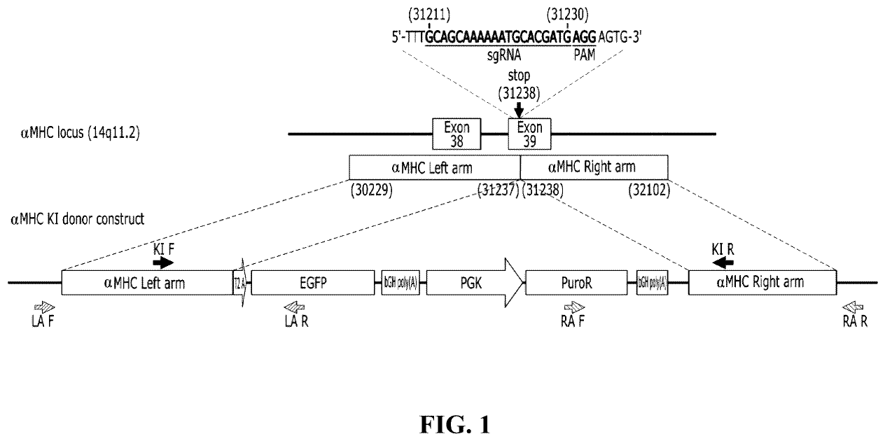

The genetic engineering may be performed using the genetic scissors CRISPR/Cas9 system. The CRISPR/Cas9 system may be composed of a Cas9 plasmid, sgRNA, and a donor construct. The sgRNA may target exon 39 of the α-MHC gene, and the donor construct may be composed of an insertion sequence containing the fluorescent reporter gene between the two homologous arm sequences of the α-MHC gene. This is schematically shown in FIG. 1.

The Cas9 plasmid, the sgRNA and the donor construct may be delivered into human pluripotent cells. For intracellular delivery, electroporation, lipofection, a viral vector, nanoparticles, or a protein translocation domain (PTD) fusion protein method may be used, without being limited thereto. Preferably, electroporation may be used.

The genetically engineered human pluripotent cell line may be produced by genetically engineering a human embryonic stem cell line or a human induced pluripotent stem cell line. Preferably, the human embryonic stem cell line SNUhES3 may be used.

In addition, according to another aspect of the present invention, there may be provided a method of monitoring differentiation into cardiomyocytes using the genetically engineered human pluripotent cell line.

For example, when the genetically engineered human pluripotent cell line is differentiated into cardiomyocytes, α-MHC may be expressed in the early stage of cardiomyocyte differentiation, and thus the fluorescent reporter may be expressed. Accordingly, it is possible to check in real time whether differentiation is proceeding properly using a fluorescence microscope, and to select only properly differentiated cells using a flow cytometer.

Hereinafter, the present invention will be described in more detail by way of examples and test examples. However, the following examples and test examples are intended to illustrate the present invention, and the scope of the present invention is not limited thereto.

### EXAMPLES

### 1. Cell Culture

The human embryonic stem cell line SNUhES3 was cultured on a vitronectin-coated plate using Essential 8 (E8, Gibco) medium under feeder-free conditions. Subculture was performed at a ratio of 1:20 after detaching the cells using accutase.

### 2. Production of α-MHC-EGFP Reporter Cell Line

SNUhES3 cells (1×10⁵) were electroporated with a donor construct, a Cas9 (Type II-A Streptococcus pyogenes Cas9, ToolGen) plasmid, and a sgRNA (ToolGen) plasmid using the Neon transfection system (Thermo Fisher Scientific). The gRNA targets exon 39 of the α-MHC gene, and the donor construct contains a T2A cleavage sequence, an EGFP gene sequence, and a puromycin resistance gene sequence between the two homologous arm sequences. This is shown in FIG. 1.

After electroporation, the cells were plated on a vitronectin-coated plate using E8 medium supplemented with Y-27632. After culturing for about 14 days, 16 colonies resistant to puromycin were isolated by puromycin selection and cultured separately. Thereafter, genomic DNA (gDNA) was extracted from each colony and subjected to PCR to confirm whether the genetic engineering was performed properly. gDNA was extracted using the DNeasy blood & Tissue kit (Qiagen), and PCR was performed using EmeraldAmp® GT PCR Master Mix (TAKARA Bio Inc.) and TaKaRa LA Taq® DNA polymerase (TAKARA Bio Inc.). Primer positions are shown in FIG. 1.

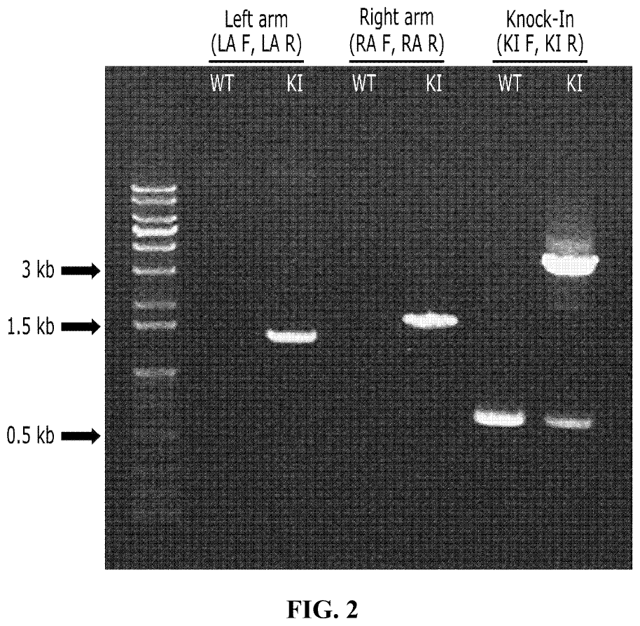

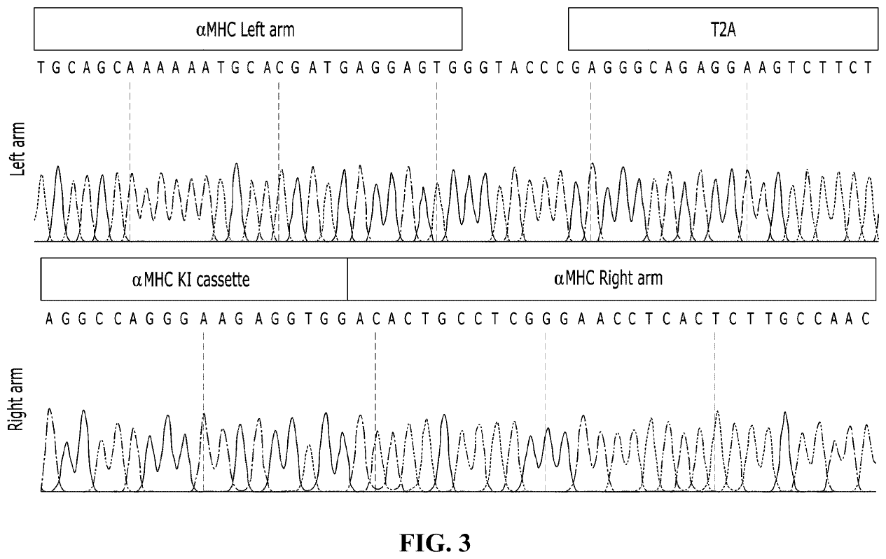

Clones in which only one chromosome was genetically engineered were selected through PCR and subjected to DNA sequencing. The results of PCR are shown in FIG. 2, and the results of DNA sequencing are shown in FIG. 3. Thereby, the SNUe003-A-3 cell line in which the EGFP gene was inserted following the α-MHC gene was produced.

### 3. Validation of SNUe003-A-3 Cell Line

It was checked whether the SNUe003-A-3 cell line would be damaged during the genetic engineering process and would maintain its pluripotency, and whether EGFP would be co-expressed with α-MHC during differentiation into cardiomyocytes.

### 3-1) Karyotype Analysis and STR Analysis

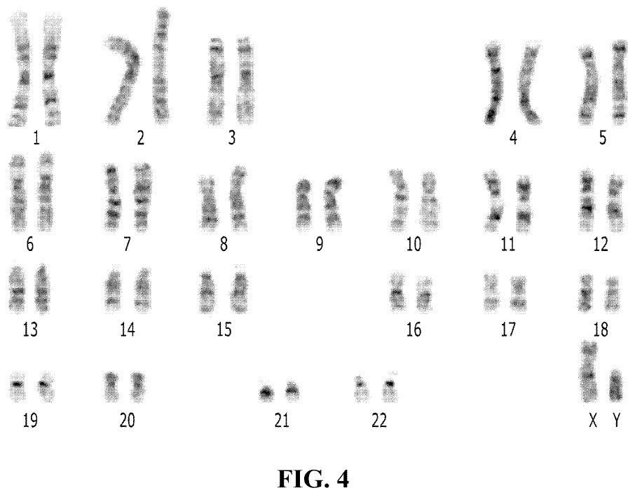

Karyotype analysis was performed using a standard protocol for GTG banding, and STR analysis was performed by DowGene Co., Ltd. The results are shown in FIG. 4, and it was confirmed that there was no damage to the karyotype.

### 3-2) Mycoplasma Contamination Analysis

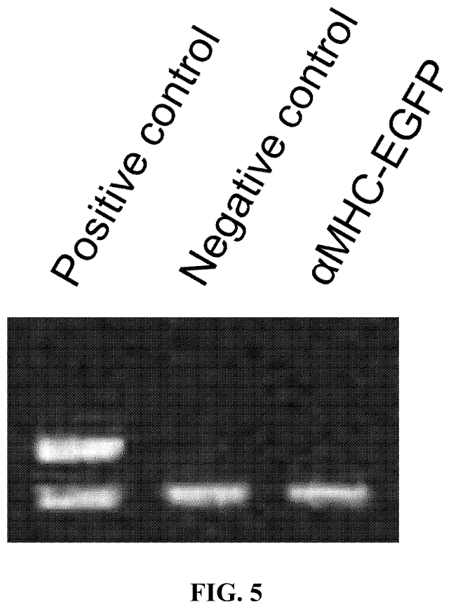

The presence of Mycoplasma contamination was checked using the e-Myco™ Mycoplasma PCR detection kit (iNtRON Biosciences, Inc.) and the results are shown in FIG. 5. It was confirmed that there was no Mycoplasma contamination in the SNUe003-A-3 cell line.

### 3-3) Confirmation of Maintenance of Pluripotency

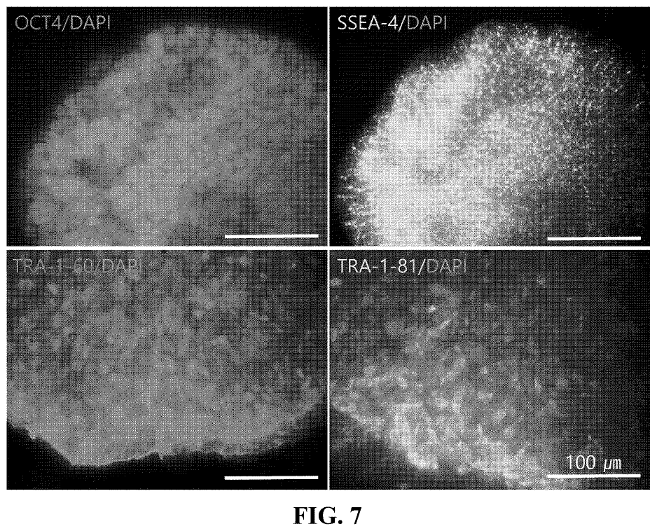

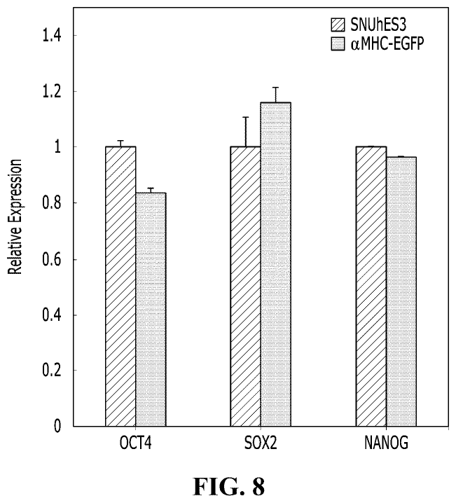

The SNUe003-A-3 cell line showed a typical human embryonic stem cell morphology and had AP activity, and the results are shown in FIG. 5. In addition, the expression of pluripotency markers OCT4, SSEA-4, TRA-1-60, and TRA-1-81 was analyzed through fluorescent staining, and the results are shown in FIG. 7. The expression of pluripotency markers OCT4, SOX2, and NANOG was analyzed through RT-PCR, and the results are shown in FIG. 8. From these results, it was confirmed that the pluripotency of the SNUe003-A-3 cell line was not damaged during the genetic engineering process.

3-4) Experiment on Differentiation into Three Germ Layers

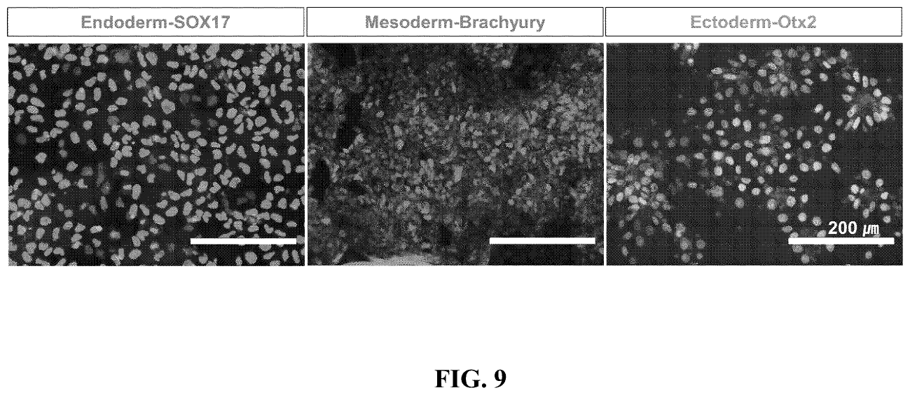

To confirm whether the SNUe003-A-3 cell line can differentiate into the three germ layers, the cell line was differentiated into the three germ layers using the Human Pluripotent Stem Cell Functional Identification kit (R&D System), and then the expression of markers for each germ layer was analyzed through immunofluorescence staining. It was confirmed that the endoderm marker SOX17, the mesoderm marker Brachyury, and the ectoderm marker OTX2 were expressed. The results are shown in FIG. 9.

3-5) Differentiation into Cardiomyocytes

It was checked whether α-MHC and EGFP would be co-expressed when the SNUe003-A-3 cell line was differentiated into cardiomyocytes. Differentiation into cardiomyocytes was performed using a slight modification of the cardiomyocyte differentiation kit (Gibco).

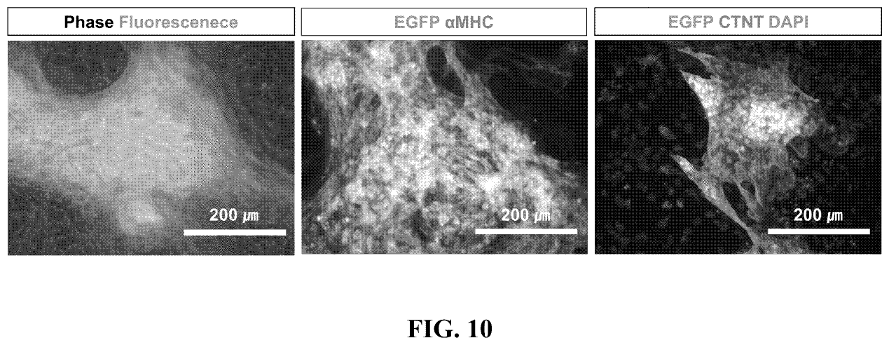

Green fluorescence and beating activity were observed from day 8 of differentiation. Whether the cells expressing green fluorescence co-express α-MHC and CTNT, another cardiomyocyte marker, was analyzed through fluorescence staining. It was confirmed that the cells expressing green fluorescence all co-expressed α-MHC and CTNT. The results are shown in FIG. 10.

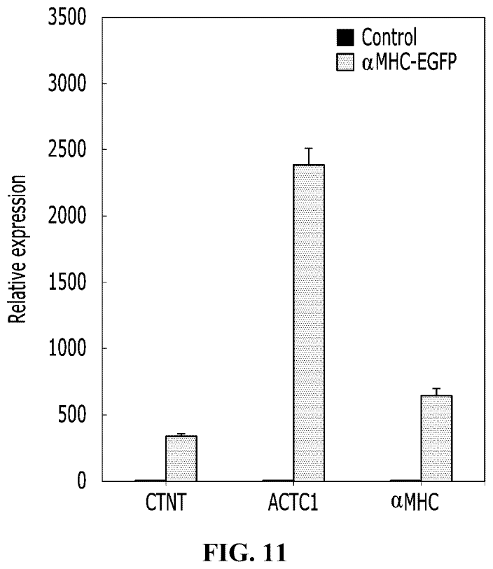

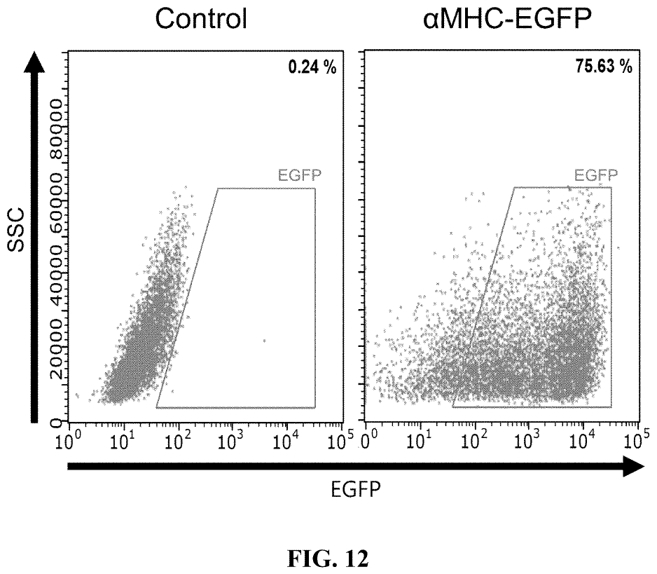

Additionally, it was confirmed through the qPCR experiment that the EGFP-expressing cells had higher expression levels of α-MHC and other cardiomyocyte markers, CTNT and ACTC1, compared to the control group, and the results are shown in FIG. 11. In addition, it was confirmed by flow cytometry analysis that 75.63% of the EGFP-expressing cells expressed α-MHC. The results are shown in FIG. 12.

Thereby, it was confirmed that the SNUe003-A-3 cell line co-expressed α-MHC and the EGFP reporter during differentiation into cardiomyocytes, indicating that differentiation of the SNUe003-A-3 cell line into cardiomyocytes could be monitored through expression of the EGFP reporter.

The antibodies used for fluorescent staining and the kit used for AP staining are shown in Table 1 below.

TABLE 1

| | Dilution | |
|---|---|---|
| Antibody/kit | ratio | Source (cat#) |
| Rabbit anti-OCT3/4 | 1:100 | Santa Cruz, Cat#sc-9081 |
| Mouse anti-SSEA4 | 1:100 | Millipore, MAB4304 |
| Mouse anti-TRA-1-60 | 1:100 | Millipore, MAB4360 |
| Mouse anti-TRA-1-81 | 1:100 | Millipore, MAB4381 |
| Alkaline phosphatase | | Sigma-Aldrich, Cat#86R-1KT |
| α-MHC | 1:200 | R&D Systems, Cat#MAB8979 |
| CTNT | 1:200 | Invitrogen, Cat#MA5-12960 |
| Goat anti-human OTX2 | 1:200 | R&D Systems, Cat#AF1979 |
| Goat anti-human | 1:200 | R&D Systems, Cat#AF2085 |
| BRACHYURY | | |
| Goat anti-human SOX17 | 1:200 | R&D Systems, Cat#AF1924 |
| Alexa Fluor 594 donkey | 1:200 | Thermo Fisher Scientific, |
| anti-goat IgG | | Cat#A11058 |
| Alexa Fluor 488 donkey | 1:200 | Thermo Fisher Scientific, |
| anti-goat IgG | | Cat#A11055 |
| Alexa Fluor 594 donkey | 1:200 | Thermo Fisher Scientific, |
| anti-rabbit IgG | | Cat#A21207 |
| Alexa Fluor 488 donkey | 1:200 | Thermo Fisher Scientific, |
| anti-mouse IgG | | Cat#A21202 |
| Alexa Fluor 488 donkey | 1:200 | Thermo Fisher Scientific, |
| anti-mouse IgG | | Cat#A21203 |

The above description of the present invention is exemplary, and those of ordinary skill in the art to which the present invention pertains will appreciate that the present invention can be easily modified into other specific forms without departing from the technical spirit or essential characteristics of the present invention. Therefore, it should be understood that the exemplary embodiments described above are exemplary in all aspects and are not restrictive. For example, each component described to be of a single type can be implemented in a distributed manner. Likewise, components described to be distributed can be implemented in a combined manner.

The scope of the present invention is defined by the following claims, and it shall be understood that all modifications and embodiments conceived from the meaning and scope of the claims and their equivalents are included in the scope of the present invention.

### MODE FOR INVENTION

The mode for carrying out the invention has been described in the “Best Mode” section.

### INDUSTRIAL APPLICABILITY

The cell line produced according to one embodiment of the present invention makes it possible to monitor whether differentiation into cardiomyocytes is progressing properly by checking the fluorescence signal of the fluorescence reporter during differentiation into cardiomyocytes. Accordingly, the cell line may be used for the development of cell therapy products for heart diseases or drug screening for the development of therapeutic agents, and thus is highly industrially applicable.

1. A genetically engineered human pluripotent cell line wherein an insertion sequence comprising a fluorescent reporter gene sequence is inserted following a stop codon of an α-MHC gene so that the α-MHC gene and the fluorescent reporter gene are co-expressed.

2. The genetically engineered human pluripotent cell line according to claim 1, wherein the fluorescent reporter is selected from the group consisting of Sirius, EBFP, ECFP, mTurquoise, TagCFP, AmCyan, mTFP1, MidoriishiCyan, CFP, TurboGFP, AcGFP, TagGFP, Azami-Green, ZsGreen, EmGFP, EGFP, GFP2, HyPer, TagYFP, EYFP, Venus, YFP, PhiYFP, PhiYFP-m, TurboYFP, ZsYellow, mBanana, KusabiraOrange, mOrange, TurboRFP, DsRed-Express, DsRed2, TagRFP, DsRed-Monomer, AsRed2, mStrawberry, TurboFP602, mRFP1, JRed, KillerRed, mCherry, HcRed, KeimaRed, mRasberry, mPlum, PS-CFP, Dendra2, Kaede, EosFP, and KikumeGR.

3. The genetically engineered human pluripotent cell line according to claim 1, wherein the insertion sequence comprise a T2A cleavage sequence, the fluorescent reporter gene sequence, and a puromycin resistance gene sequence.

4. The genetically engineered human pluripotent cell line according to claim 3, wherein the insertion sequence comprises SEQ ID NO: 1.

5. The genetically engineered human pluripotent cell line according to claim 3, wherein the α-MHC and fluorescent reporter genes are translated into their respective proteins by the T2A cleavage sequence.

6. The genetically engineered human pluripotent cell line according to claim 1, wherein the genetic engineering is achieved by a CRISPR/Cas9 system.

7. The genetically engineered human pluripotent cell line according to claim 1, wherein the human pluripotent cell line is produced by genetically engineering a human embryonic stem cell line, SNUhES3.

8. The genetically engineered human pluripotent cell line according to claim 1, wherein the genetically engineered human pluripotent cell line expresses fluorescence while differentiating into cardiomyocytes.

9. A method of monitoring differentiation into cardiomyocytes using the genetically engineered human pluripotent cell line according to claim 1.
